(19) United States
(12) Patent Application Publication
(10) Pub. No.: US 2020/0352844 A1
(43) Pub. Date: Nov. 12, 2020

(54) COMPOSITION WITH LUMINOUS SATIN EFFECT COMPRISING PARTICLES OF CELLULOSE, OF BORON NITRIDE AND OF SATIN NACRES

(71) Applicant: L'OREAL, Paris (FR)

(72) Inventors: Ludovic THEVENET, Chevilly la Rue (FR); Khonesavanh CHANSAVATH, Chevilly la Rue (FR); Olfa BCHIR, Chevilly la Rue (FR)

(73) Assignee: L'OREAL, Paris (FR)

(21) Appl. No.: 16/762,002

(22) PCT Filed: Sep. 27, 2018
(86) PCT No.: PCT/EP2018/076328

§ 371 Date: May 6, 2020

(30) Foreign Application Priority Data
Nov. 9, 2017 (FR) 1760527

(51) Int. Cl. A61K 8/73; A61K 8/19; A61K 8/98; A61K 8/02; A61K 8/06; A61Q 1/02

(52) U.S. Cl. CPC A61K 8/731; A61K 8/19; A61Q 1/02; A61K 8/0254; A61K 8/064; A61K 8/987

(57) ABSTRACT

The present invention relates to a composition, in particular comprising a physiologically acceptable medium, in particular for coating keratin materials, more particularly for caring for and/or making up keratin materials, which is a cosmetic composition for making up and/or caring for keratin materials, in particular the skin and/or the lips, comprising: a) cellulose particles; and b) boron nitride particles; and c) nacre particles having a mean size of less than 20.0 μm. The invention also relates to a process for cosmetic treatment of keratin materials, more particularly for caring for and/or making up keratin materials, such as the skin, characterized in that it comprises the application to the keratin materials of a composition as defined previously.

The present invention relates to a composition, in particular comprising a physiologically acceptable medium, in particular for coating keratin materials, more particularly for caring for and/or making up keratin materials, which is a cosmetic composition for making up and/or caring for keratin materials, in particular the skin and/or the lips, comprising:
a) cellulose particles; and
b) boron nitride particles; and
c) nacre particles having a mean size of less than 20.0 μm.

The invention also relates to a process for cosmetic treatment of keratin materials, more particularly for caring for and/or making up keratin materials, such as the skin, characterized in that it comprises the application to the keratin materials of a composition as defined previously.

The skin is not a smooth surface of even colour. It exhibits dyschromia and also reliefs and micro reliefs which form a somewhat uneven surface. These irregularities are such that the surface is sometimes judged to be unattractive.

Cosmetic care and/or makeup compositions are commonly used to camouflage and/or even up dyschromia (such as marks and redness which are diffuse or around the eyes) and/or the imperfections of the relief of the skin, such as pores, wrinkles and/or fine lines, spots, acne marks and/or scars. In this regard, many solid or fluid, anhydrous or non-anhydrous formulations have been developed to date.

In order to obtain this skin imperfection-correcting effect, coverage is one of the main properties desired. For this purpose, care and/or makeup products which use pigments based on metal oxides such as iron oxides and/or titanium oxides that can be modified with a surface-treatment agent are often used. However, the latter are opaque, and have a tendency to accumulate in the reliefs such as pores and wrinkles, to create a contrast in terms of opacity and colour and to accentuate these relief imperfections.

In order to improve the correction performance levels and to reduce this accentuation, it is known practice to use nacres often combined with soft-focus fillers, also denoted “fillers with a soft-focus effect” or else “haze-effect fillers”.

Consumers are increasingly in search of products for camouflaging and/or evening out these imperfections, that do not produce a dull effect when they are applied to the skin. On the contrary, they desire a luminous makeup result which is neither too matt nor too glossy: the term “satiny effect” will be used in the remainder of the description Excessive mattness is not aesthetic since it gives the skin an appearance of dryness and a powdered effect that does not bring out the features of the face. At the opposite end of the scale, excessive gloss is not desired either, since this result is associated with the presence of sebum or sweat on the skin. It can also give the impression of poor health (greasy film on the face which does not allow the skin to breathe) and accentuates the visibility of the relief imperfections.

However, the products intended to correct imperfections that are currently on the market and that contain nacres generally combined with soft-focus fillers do not generally make it possible to obtain at the same time a good camouflage effect and a luminous satin effect. Indeed, the presence of soft-focus fillers has a tendency to result in a finish that is too matt and in the absence of a luminous effect. The use of nacres makes it possible to provide light but can produce an effect that is too iridescent and can give an unnatural and artificial sparkling appearance. Furthermore, said nacres can pose problems of uniformity which do not make it possible to produce the desired effect of light.

There therefore remains a need to find new care and/or makeup compositions based on a combination of suitable nacres and soft-focus fillers making it possible to camouflage skin imperfections without the drawbacks previously mentioned and to obtain both a soft-focus effect which is not dull and a luminous satin effect.

During its research, the applicant has discovered, surprisingly, that this objective is obtained by virtue of a composition, in particular comprising a physiologically acceptable medium, in particular for coating keratin materials, more particularly for caring for and/or making up keratin materials, which is a cosmetic composition for making up and/or caring for keratin materials, in particular the skin and/or the lips, comprising:
a) cellulose particles; and
b) boron nitride particles; and
c) nacre particles having a mean size of less than 20.0 μm.

This discovery forms the basis of the invention.

The present invention relates to a composition, in particular comprising a physiologically acceptable medium, in particular for coating keratin materials, more particularly for caring for and/or making up keratin materials, which is a cosmetic composition for making up and/or caring for keratin materials, in particular the skin and/or the lips, comprising:
a) cellulose particles; and
b) boron nitride particles; and
c) nacre particles having a mean size of less than 20.0 μm.

The invention also relates to a process for cosmetic treatment of keratin materials, more particularly for caring for and/or making up keratin materials, such as the skin, characterized in that it comprises the application to the keratin materials of a composition as defined previously.

### Definitions

In the context of the present invention, the term “keratin materials” is intended to mean the skin and more particularly the areas such as the face, the neck, the lips, the cheeks, the hands, the body, the legs and the thighs, the area around the eyes, and the eyelids.

The term “physiologically acceptable” is intended to mean compatible with the skin and/or its integuments, which has a pleasant colour, odour and feel and which does not give rise to any unacceptable discomfort.

For the purposes of the invention, the term “mean” particle “size” is intended to mean the median value D[50] representing the maximum size of 50% by volume of the particles. Said mean particle size is measured at 25° C. according to the static light scattering particle-sizing method using a particle size analyser such as the Mastersizer 2000 from Malvern or of the Microtrac type from Nikkiso. The light intensity scattered by the particles as a function of the angle at which they are illuminated is converted to size distribution according to the Mie theory. This theory is particularly described in the publication by Van de Hulst, H. C., Light Scattering by Small Particles, Chapters 9 and 10, Wiley, New York, 1957.

According to the invention, the term “satin effect” is intended to mean that the formula reflects light mainly in the specular direction and uniformly over the entire evaluation support or on the face and that this light is not too intense like the gloss of a glossy oil or like the sparkling iridescent or nacreous effect of certain nacres which can give a rendering associated with makeup more than with care. If, on the other hand, the formula does not reflect light, the formula has a “matt” rendering. The satin effect is linked to a directional diffuse surface reflection. For a given angle of incidence, the directional diffuse reflection has a maximum close to the specular direction and a diffuse lobe surrounding it. The rougher the surface, the less intense the surface reflection in the specular direction, and the formula becomes matt and the light is scattered in all directions.

It is possible to evaluate this satin effect by means of sensory tests carried out on a panel of experienced women who know how to distinguish the mat effect from glossy effects and know how to categorise the various types of gloss (satin effect, nacreous effect, effect which is iridescent, oily glossy, etc.).

The term “luminous or radiant effect” is intended to mean women between 30 and 60 years old who have a smooth, even skin without relief imperfections and having a satin effect which reflects back light. It is possible to evaluate this satin effect by means of sensory tests carried out on a panel of experienced women who know how to evaluate this glowing, light or radiance effect, following the application of the formulas.

The soft-focus correction effect is characterized by haze and transparency (transmittance TH) measurements. “Haze” corresponds to the percentage of light scattered relative to the total transmittance according to standard ASTM D 1003 (Standard Test Method for Haze and Luminous Transmittance of Transparent Plastics).

25 μm films of composition are applied to 50 μm polyethylene (PE) films. The film is then measured after 1 hour of drying at ambient temperature (25° C.). Finally, the film is placed in the machine and transparency and haze measurements are taken.

According to one preferred form of the invention, the composition is characterized in that the Haze is greater than 80% and the transmission TH is greater than 80%, and more preferentially the Haze is greater than 90% and the transmission TH is greater than 90% according to standard ASTM D 1003 (Standard Test Method for Haze and Luminous Transmittance of Transparent Plastics).

### Cellulose Particles

The cellulose particles that may be used according to the invention are preferably spherical (cellulose beads).

For the purposes of the present invention, the term “spherical particles” is intended to mean solid or porous particles which have a circularity parameter of at least 0.95. The circularity parameter is defined as the ratio of the circumference of a disc having the same area as the particle to the perimeter of the particle. A value of 1 characterizes perfectly spherical particles.

They preferably have a mean size of less than 40 μm, preferably ranging from 1 to 20 μm, more preferentially from 2 to 10 μm.

Among the cellulose particles that may be used according to the invention, mention may in particular be made of those sold by the company Daito under the brand name Cellulosbeads® such as Cellulobeads USF® (D[50]=4 μm), Cellulobeads D-5® (D[50]<10 μm), Cellulobeads D-10® (D[50]<15 μm) or Cellulobeads D-30® (D[50]<30 μm).

Preferably, the cellulose particles are present in a content ranging from 0.5% to 10% by weight, more preferentially from 1% to 6% by weight, relative to the total weight of the composition.

### Boron Nitride Particles

There are several polymorphic forms of boron nitride: hexagonal form boron nitrides (denoted h-BN) or wurzite-type hexagonal form boron nitrides (denoted w-BN), rhombohedral form boron nitrides (denoted r-BN), amorphous form boron nitrides (denoted a-BN), turbostatic boron nitrides (denoted t-BN) and cubic form boron nitrides (denoted c-BN). They may be of diverse forms, including the platelet form.

According to one particular form of the invention, the boron nitride particles have a platelet form.

Preferentially, the boron nitride particles have a mean size of greater than 5 μm, preferably ranging from 5 to 50 μm, and more preferentially from 5 to 20 μm.

Preferably, the boron nitride particles are present in a content ranging from 0.1% to 5% by weight, more preferentially from 0.3% to 2.5% by weight, relative to the total weight of the composition.

The boron nitride particles in accordance with the invention will be chosen more particularly from the following commercial products:

SOFTOUCH BORON NITRIDE CC6058® from the company MOMENTIVE,
SOFTOUCH BORON NITRIDE CC6059® from the company MOMENTIVE,
SOFTOUCH BORON NITRIDE CC6004® from the company MOMENTIVE,
SOFTOUCH BORON NITRIDE CC6064® from the company MOMENTIVE,
SOFTOUCH BORON NITRIDE CC6069® from the company MOMENTIVE,
SOFTOUCH BORON NITRIDE CCS102® from the company MOMENTIVE,
SOFTOUCH BORON NITRIDE CCS402® from the company MOMENTIVE,
PUHP 3008 from the company SAINT GOBAIN CERAMICS,
RONAFLAIR BORONEIGE SQ-6 from the company MERCK.

The SOFTOUCH BORON NITRIDE CCS102® product will more particularly be used.

### Nacre Particles

In the context of the present invention, the term “nacres” should be understood to mean white or bulk-coloured interference multilayer particles which have an interference colour and which can have various levels of sparkle.

The nacre particles according to the invention are composite particles constituted of several materials. The particles are in platelet form.

The nacres generally have a multilayer structure constituted of a natural or synthetic substrate coated with one or more layers of a material different from the substrate.

These particles are therefore constituted of several materials. They thus comprise a base layer corresponding to the substrate on which is superimposed at least one layer of another material. The substrate according to the invention can be covered for example with one, two or three distinct layers of different nature.

The nacres can be chosen from:

i) natural or synthetic micas (fluorphlogopite) covered with at least one layer of titanium dioxide (titanium mica), in particular said titanium micas being covered with at least one material chosen from iron oxides, bismuth oxychloride, chromium oxide, and organic dyes such as organic pigments of the lake type. They may in particular be mica particles, at the surface of which are superposed at least two successive layers of metal oxides and/or of organic colorants;
ii) natural or synthetic micas covered with at least one layer of bismuth oxychloride (bismuth oxychloride-mica), in particular said bismuth oxychloride-micas being covered with at least one material chosen from iron oxides, chromium oxide, chromium hydroxide, ultramarines, and organic dyes such as organic pigments of the lake type;
iii) nacres having silica or alumina as substrate, said substrate being covered with at least one layer constituted of at least one metal oxide, in particular chosen from titanium oxides and iron oxides;
iv) mixtures thereof.

According to one preferential embodiment, use will be made of nacre particles comprising a substrate based on natural or synthetic mica (fluorphlogopite) covered with at least one layer constituted of at least one metal oxide chosen from titanium oxides, iron oxides, tin oxides, and mixtures thereof.

Among the nacres available on the market, mention may be made of the nacres having the following brand names:

TIMIRON SILK, XIRONA® and COLORONA® from the company MERCK,

SUNSHINE FINE, SPECTRAFLEX® and INTENZA PASSIONATE KISS from the company SUN CHEMICAL,

HELIOS® from the company TOPY,

SYNCRYSTAL® from the company ECKART,

FLAMENCO SATIN®, TIMICA®, BI-LITE® and CHROMA-LITE® from the company BASF.

In particular, use will be made of the following nacres having the following INCI names and the following brand names:

TITANIUM DIOXIDE (and) MICA (TIMIRON SILK RED® from the company MERCK),

MICA (and) TITANIUM DIOXIDE (and) IRON OXIDES (COLORONA ORIENTAL BEIGE® from the company MERCK),

SYNTHETIC FLUORPHLOGOPITE (and) TITANIUM DIOXIDE (SUNSHINE FINE WHITE (C80-3100)® from the company SUN CHEMICAL),

TITANIUM DIOXIDE (and) SYNTHETIC FLUORPHLOGOPITE (and) TIN OXIDE (SYNCRYSTAL RED® from the company ECKART),

TITANIUM DIOXIDE (and) SYNTHETIC FLUORPHLOGOPITE (and) RED 7 LAKE (INTENZA PASSIONATE KISS C91-4131® from the company SUN CHEMICAL), MICA (and) TITANIUM DIOXIDE (TIMICA TERRA WHITE MN4501® from the company BASF),

IRON OXIDES (and) SILICA (XIRONA LE ROUGE© from the company MERCK), ALUMINA (and) TITANIUM DIOXIDE (SPECTRAFLEX FOCUS BLUEC88-1051®, SPECTRAFLEX FOCUS GOLDC88-1011®. SPECTRAFLEX FOCUS REDC88-1031®, SPECTRAFLEX FOCUS WHITEC88-1001® from the company SUN CHEMICAL),

MICA (and) BISMUTH OXYCHLORIDE (BI-LITE 20® from the company BASF)

MICA (and) BISMUTH OXYCHLORIDE (and) IRON OXIDES (CHROMA-LITE BLACK CL4498®, CHROMA-LITE BRONZE CL4499®, CHROMA-LITE YELLOW CL4502®, CHROMA-LITE GOLD CL4504®, CHROMA-LITE RED CL4506®, CHROMA-LITE BROWN CL4509®, CHROMA-LITE MAUVE CL4511® from the company BASF),

MICA (and) BISMUTH OXYCHLORIDE (and) FERRIC FERROCYANIDE (CHROMA-LITE DARK BLUE CL4501® from the company BASF),

MICA (and) BISMUTH OXYCHLORIDE (and) CHROME OXIDE (CHROMA-LITE GREEN CL4503® from the company BASF

MICA (and) BISMUTH OXYCHLORIDE (and) CARMINE (CHROMA-LITE MAGENTA CL4505® from the company BASF

MICA (and) BISMUTH OXYCHLORIDE (and) MANGANESE VIOLET (CHROMA-LITE VIOLET CL4507® from the company BASF)

MICA (and) BISMUTH OXYCHLORIDE (and) ULTRAMARINE BLUE (CHROMA-LITE PURPLE CL4510® from the company BASF).

The nacre particles according to the invention have a mean size of less than 20.0 μm.

Preferably, the nacre particles are present in a content ranging from 0.1% to 3% by weight, more preferentially from 0.1% to 2% by weight, relative to the total weight of the composition.

### Presentation Forms

The composition according to the invention may in particular be in the form of an aqueous solution, aqueous-alcoholic solution or oily solution, it being possible for said solution to be gelled, a dispersion of the lotion type, which is optionally a two-phase or three-phase lotion, an oil-in-water or water-in-oil or multiple emulsion, it being possible for said emulsions to be optionally gelled, an aqueous or anhydrous gel, a dispersion of oil(s) in an aqueous phase with the aid of spherules, it being possible for these spherules to be polymer particles or, better still, lipid vesicles of ionic and/or non-ionic type, a serum, a paste, or a soft or rigid stick or rod which can melt on the skin or semi-mucous membranes. It may be of solid, pasty or more or less fluid liquid consistency.

According to one preferential form, the compositions are in the form of a water-in-oil emulsion comprising a continuous oily phase in which an aqueous phase is dispersed.

### A) Aqueous Phase

The aqueous phase comprises water and optionally water-soluble or water-miscible ingredients, such as water-soluble solvents.

A water that is suitable for use in the invention may be a floral water such as cornflower water and/or a mineral water such as Vittel water, Lucas water or La Roche Posay water and/or a thermal spring water.

The water may be present in the composition according to the invention in a content ranging from 5% to 80% by weight, more preferentially from 10% to 70% by weight and more preferentially ranging from 20% to 70% by weight, relative to the total weight of said composition.

In the present invention, the term “water-soluble solvent” denotes a compound that is liquid at ambient temperature and water-miscible (miscibility in water of greater than 50% by weight at 25° C. and atmospheric pressure).

The water-soluble solvents that may be used in the composition of the invention may also be volatile.

Among the water-soluble solvents that may be used in the composition in accordance with the invention, mention may be made especially of lower monoalcohols containing from 1 to 5 carbon atoms such as ethanol and isopropanol, glycols containing from 2 to 8 carbon atoms such as ethylene glycol, propylene glycol, 1,3-butylene glycol and dipropylene glycol, C₃ and C₄ ketones and C₂-C₄ aldehydes.

When the composition is a water-in-oil emulsion, the aqueous phase is preferably present in a concentration ranging from 5% to 88% by weight, preferably ranging from 20% to 85% by weight, relative to the total weight of said composition.

### B) Oily Phase

The emulsion of the invention also comprises an oily phase. Said phase is liquid (in the absence of structuring agent) at ambient temperature (20-25° C.). Preferentially, the water-immiscible organic liquid phase in accordance with the invention generally comprises at least one volatile oil and/or one non-volatile oil and optionally any ingredient that is soluble or miscible in the oily phase.

The term “oil” is intended to mean a fatty substance which is liquid at ambient temperature (25° C.) and atmospheric pressure (760 mmHg, i.e. 105 Pa). The oil may be volatile or non-volatile.

For the purposes of the invention, the term “volatile oil” refers to an oil that is capable of evaporating on contact with the skin or the keratin fibre in less than one hour, at ambient temperature and atmospheric pressure. The volatile oils of the invention are volatile cosmetic oils which are liquid at ambient temperature, having a non-zero vapour pressure, at ambient temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

The term “non-volatile oil” is intended to mean an oil that remains on the skin or the keratin fibre at ambient temperature and atmospheric pressure for at least several hours, and that especially has a vapour pressure of less than 10-mmHg (0.13 Pa).

The oil may be chosen from any oil, which is preferably a physiologically acceptable oil, in particular mineral, animal, plant or synthetic oils; in particular volatile or non-volatile hydrocarbon-based oils and/or silicone oils and/or fluoro oils, and mixtures thereof.

More precisely, the term “hydrocarbon-based oil” is intended to mean an oil mainly comprising carbon and hydrogen atoms and optionally one or more functions chosen from hydroxyl, ester, ether and carboxylic functions. Generally, the oil has a viscosity of from 0.5 to 100 000 mPa·s, preferably from 50 to 50 000 mPa·s and more preferably from 100 to 300 000 mPa·s.

For the purposes of the present invention, the term “silicone oil” is intended to mean an oil comprising at least one silicon atom, and especially at least one Si—O group.

For the purposes of the present invention, the term “fluoro oil” is intended to mean an oil comprising at least one fluorine atom.

The concentration of oily phase of the emulsion of the invention preferably ranges from 3% to 90% by weight, more particularly ranging from 10% to 80% by weight, relative to the total weight of the composition.

As examples of volatile hydrocarbon-based oils that may be used in the invention, mention may be made of:
hydrocarbon-based oils containing from 8 to 16 carbon atoms, and especially C₈-C₁₆ isoalkanes of petroleum origin (also known as isoparaffins), for instance isododecane (also known as 2,2,4,4,6-pentamethylheptane), isodecane and isohexadecane, for example the oils sold under the trade names Isopar or Permethyl, branched C₈-C₁₆ esters and isohexyl neopentanoate, and mixtures thereof. Other volatile hydrocarbon-based oils, for instance petroleum distillates, especially those sold under the name Shell Solt by the company Shell, may also be used; volatile linear alkanes, such as those described in patent application DE10 2008 012 457 from the company Cognis.

By way of example of a volatile silicone oil that can be used in the invention, mention may be made of volatile silicone oils, for instance linear or cyclic volatile silicone oils, in particular those having a viscosity 8 centistokes (8×10⁶ m²/s),

and especially having from 2 to 7 silicon atoms, these silicones optionally comprising alkyl or alkoxy groups containing from 1 to 10 carbon atoms. As volatile silicone oils that may be used in the invention, mention may be made in particular of caprylyl methicone, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane;
and mixtures thereof.

As examples of non-volatile hydrocarbon-based oils that may be used in the invention, mention may be made of:
hydrocarbon-based oils of animal origin, such as perhydrosqualene;
linear or branched hydrocarbons, of mineral or synthetic origin, such as liquid paraffins and derivatives thereof, petroleum jelly, polydecenes, polybutenes, hydrogenated polyisobutene such as Parleam, or squalane;
phytostearyl esters, such as phytostearyl oleate, phytostearyl isostearate and lauroyl/octyldodecyl/phytostearyl glutamate (Ajinomoto, Eldew PS203®);
triglycerides constituted of fatty acid esters of glycerol, the fatty acids of which may in particular have chain lengths ranging from C₄ to C₃₆, and especially from C₁₈ to C₃₆, these oils possibly being linear or branched, and saturated or unsaturated; these oils may especially be heptanoic or octanoic triglycerides, wheatgerm oil, sunflower oil, grapeseed oil, sesame seed oil (820.6 g/mol), corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passionflower oil or musk rose oil; shea oil; or alternatively caprylic/capric acid triglycerides, for instance those sold by the company Stéarineries Dubois or those sold under the names Miglyol 810®, 812® and 818® by the company Dynamit Nobel;
synthetic ethers containing from 10 to 40 carbon atoms, such as dicaprylyl ether;
hydrocarbon-based esters of formula RCOOR′ in which RCOO represents a carboxylic acid residue comprising from 2 to 40 carbon atoms, and R′ represents a hydrocarbon-based chain containing from 1 to 40 carbon atoms, such as cetostearyl octanoate, isopropyl alcohol esters, such as isopropyl myristate or isopropyl palmitate, ethyl palmitate, 2-ethylhexyl palmitate, isopropyl stearate or isostearate, isostearyl isostearate, octyl stearate, diisopropyl adipate, heptanoates, and in particular isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate and palmitate, alkyl benzoate, polyethylene glycol diheptanoate, propylene glycol 2-diethyl hexanoate, and mixtures thereof, C12 to C15 alcohol benzoates, hexyl laurate, neopentanoic acid esters, for instance isodecyl neopentanoate, isotridecyl neopentanoate, isostearyl neopentanoate and 2-octyldodecyl neopentanoate, isononanoic acid esters, for instance isononyl isononanoate, isotridecyl isononanoate and octyl isononanoate, oleyl erucate, isopropyl lauroyl sarcosinate, diisopropyl sebacate, isocetyl stearate, isodecyl neopentanoate, isostearyl behenate, and myristyl myristate;
polyesters obtained by condensation of unsaturated fatty acid dimer and/or trimer and of diol, such as those described in patent application FR 0 853 634, in particular such as dilinoleic acid and 1,4-butanediol. Mention may in particular be made in this respect of the polymer sold by Biosynthis under the name Viscoplast 14436H® (INCI name: Dilinoleic Acid/Butanediol Copolymer), or copolymers of polyols and of diacid dimers, and esters thereof, such as Hailuscent ISDA®,
polyol esters and pentaerythritol esters, for instance dipentaerythritol tetrahydroxystearate/tetraisostearate,
fatty alcohols containing from 12 to 26 carbon atoms, for instance octyldodecanol, 2-butyloctanol, 2-hexyldecanol, 2-undecylpentadecanol and oleyl alcohol;
dialkyl carbonates, the two alkyl chains possibly being identical or different, such as dicaprylyl carbonate sold under the name Cetiol CC® by Cognis, and
vinylpyrrolidone copolymers such as the vinylpyrrolidone/1-hexadecene copolymer, Antaron V-216® sold or manufactured by the company ISP,
linear fatty acid esters with a total carbon number ranging from 35 to 70, such as pentaerythrityl tetrapelargonate,
hydroxylated esters such as polyglyceryl-2 triisostearate;
aromatic esters such as tridecyl trimellitate, C₁₂-C₁₅ alcohol benzoates, the 2-phenylethyl ester of benzoic acid, and butyloctyl salicylate,
C₂₄-C₂₈ esters of branched fatty alcohols or fatty acids such as those described in patent application EP-A-0 955 039, and in particular triisoarachidyl citrate, pentaerythrityl tetraisononanoate, glyceryl triisostearate, glyceryl tris(2-decyl)tetradecanoate, pentaerythrityl tetraisostearate, polyglyceryl-2 tetraisostearateorpentaerythrityltetrakis(2-decyl)tetradecanoate,
esters and polyesters of dimer diol and of monocarboxylic or dicarboxylic acid, such as esters of dimer diol and of fatty acid and esters of dimer diol and of dimer dicarboxylic acid, such as Lusplan DD-DA5® and Lusplan DD-DA7® sold by the company Nippon Fine Chemical and described in patent application US 2004-175 338, the content of which is incorporated into the present application by reference,
and mixtures thereof.

Among the non-volatile fluoro and/or silicone oils, mention may be made of:
optionally partially hydrocarbon-based and/or silicone fluoro oils, for instance fluorosilicone oils, fluoropolyethers and fluorosilicones as described in EP-A-847 752;
silicone oils such as non-volatile polydimethylsiloxanes (PDMSs); phenylated silicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyltrimethyl-siloxysilicates.

Preferentially, the oily phase comprises at least one silicone oil, even more preferentially chosen from:
volatile cyclic silicone oils having a viscosity at ambient temperature of less than 8 cSt and containing in particular from 4 to 7 silicon atoms, these silicones optionally comprising alkyl or alkoxy groups containing from 1 to 10 carbon atoms, in particular chosen from hexamethylcyclotrisiloxane, octamethylcyclotetrasiloxane, decamethylcyclopentasiloxane and dodecamethylcyclohexasiloxane (cyclohexasiloxane), and mixtures thereof;
volatile or non-volatile polydimethylsiloxanes (PDMSs) (INCI name: Dimethicone);
phenylated silicones;
polydimethylsiloxanes comprising aliphatic groups, in particular alkyl groups, or alkoxy groups, which are pendent and/or at the end of the silicone chain; these groups each comprising from 6 to 24 carbon atoms, and more particularly caprylyl methicone, such as the commercial product Dow Corning FZ-3196® from the company Dow Corning;
mixtures thereof.

### C) Emulsifiers

The water-in-oil emulsions according to the invention generally comprise at least one water-in-oil (W/O) emulsifying surfactant, which is preferably non-ionic.

For the purposes of the present invention, “emulsifying surfactant” is intended to mean an amphiphilic surfactant compound, i.e. one which has two parts of different polarity. Generally, one is lipophilic (soluble or dispersible in an oily phase). The other is hydrophilic (soluble or dispersible in water). The emulsifying surfactants are characterized by the value of their HLB (Hydrophilic Lipophilic Balance), the HLB being the ratio between the hydrophilic part and the lipophilic part in the molecule. The term “HLB” is well known to those skilled in the art and is described, for example, in “The HLB system. A time-saving guide to Emulsifier Selection” (published by ICI Americas Inc., 1984). For the W/O emulsifying surfactants, the HLB generally ranges from 3 to 8 for the preparation of W/O emulsions. The HLB of the surfactant(s) used according to the invention may be determined via the Griffin method or the Davies method.

As examples of W/O emulsifying surfactants, mention may be made of alkyl esters or ethers of sorbitan, of glycerol, of polyol or of sugars; silicone surfactants, such as dimethicone copolyols, such as that having the INCI name Dimethicone (and) PEG/PPG-18/18 Dimethicone sold under the brand X-22-6711D® by the company Shin-Etsu, the mixture of cyclomethicone and of dimethicone copolyol, sold under the name DC 5225 C® by the company Dow Corning, and alkyldimethicone copolyols such as laurylmethicone copolyol sold under the name Dow Corning 5200 Formulation Aid by the company Dow Corning; cetyl dimethicone copolyol, such as cetyl PEG/PPG-10/1 dimethicone, such as the product sold under the name Abil EM 90® by the company Evonik Goldschmidt, and the mixture of cetyl dimethicone copolyol, of polyglyceryl isostearate (4 mol) and of hexyl laurate, sold under the name Abil WE 09® by the company Goldschmidt. One or more coemulsifiers, which may be chosen advantageously from the group comprising polyol alkyl esters, may also be added thereto.

Mention may also be made of mixtures of alkyl polyglycosides and of fatty alcohol, such as the mixture of octyldodecanol and of octyldodecyl xyloside sold under the trade name Fluidanov 20 X® from the company SEPPIC, having the INCI name Octyldodecanol (and) Octyldodecyl Xyloside.

As polyol alkyl esters, mention may be made in particular of polyethylene glycol esters, such as PEG-30 dipolyhydroxystearate, such as the product sold under the name Cithrol DPHS-SO-(MV) by the company Croda.

Examples of glycerol and/or sorbitan esters that may be mentioned include polyglyceryl isostearate (INCI name: Polyglyceryl-4 Isostearate), such as the product sold under the name Isolan GI 34® by the company Evonik Goldschmidt; sorbitan isostearate, such as the product sold under the name Arlacel 987® by the company ICI; sorbitan glyceryl isostearate, such as the product sold under the name Arlacel 986® by the company ICI, the diester of a mixture of isostearic, polyhydroxystearic and sebacic acids with Polyglycerin-4 (INCI name: Polyglyceryl-4 Diisostearate/Polyhydroxystearate/Sebacate), such as the product sold under the name Isolan GPS® by the company Evonik, and mixtures thereof.

According to a particular form of the invention, the emulsifying surfactant may be chosen from emulsifying silicone elastomers.

The term “silicone elastomer” is intended to mean a supple, deformable organopolysiloxane that has viscoelastic properties and in particular the consistency of a sponge or a supple sphere. Its modulus of elasticity is such that this material withstands deformation and has limited stretchability and contractibility. This material is capable of regaining its original shape after stretching.

The emulsifying silicone elastomer may be chosen from polyoxyalkylenated silicone elastomers and polyglycerolated silicone elastomers, and mixtures thereof.

a) Polyoxyalkylenated Silicone Elastomers

The polyoxyalkylenated silicone elastomer is a crosslinked organopolysiloxane that may be obtained by a crosslinking addition reaction of diorganopolysiloxane containing at least one hydrogen bonded to silicon and of a polyoxyalkylene containing at least two ethylenically unsaturated groups.

Preferably, the polyoxyalkylenated crosslinked organopolysiloxane is obtained by a crosslinking addition reaction (A1) of diorganopolysiloxane containing at least two hydrogens each bonded to a silicon, and (B1) of polyoxyalkylene containing at least two ethylenically unsaturated groups, in particular in the presence (C1) of a platinum catalyst, as described, for example, in U.S. Pat. Nos. 5,236,986 and 5,412,004.

In particular, the organopolysiloxane may be obtained by reaction of dimethylvinylsiloxy-terminated polyoxyalkylene (in particular polyoxyethylene and/or polyoxypropylene) and of trimethylsiloxy-terminated methylhydropolysiloxane, in the presence of a platinum catalyst.

The organic groups bonded to the silicon atoms of compound (A1) may be alkyl groups containing from 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group.

Compound (A1) may thus be chosen from trimethylsiloxy-terminated methylhydropolysiloxanes, trimethylsiloxy-terminated dimethylsiloxane/methylhydrosiloxane copolymers, dimethylsiloxane/methylhydrosiloxane cyclic copolymers, and trimethylsiloxy-terminateddimethylsiloxane/methylhydrosiloxane/laurylmethylsiloxanecopolymers.

Compound (C1) is the catalyst for the crosslinking reaction, and is in particular chloroplatinic acid, chloroplatinic acid-olefin complexes, chloroplatinic acid-alkenylsiloxane complexes, chloroplatinic acid-diketone complexes, platinum black and platinum on a support.

Advantageously, the polyoxyalkylenated silicone elastomers may be formed from divinyl compounds, in particular polyoxyalkylenes containing at least two vinyl groups, reacting with Si—H bonds of a polysiloxane.

The polyoxyalkylenated silicone elastomer according to the invention is preferably mixed with at least one hydrocarbon-based oil and/or one silicone oil to form a gel.

In these gels, the polyoxyalkylenated elastomer may be in the form of non-spherical particles.

Polyoxyalkylenated elastomers are described especially in patents U.S. Pat. Nos. 5,236,986, 5,412,004, 5,837,793 and 5,811,487.

As polyoxyalkylenated silicone elastomers, use may be made of those having the following INCI names:

### Dimethicone/PEG-10/15-Crosspolymer,

### PEG-15/Lauryl Dimethicone Crosspolymer,

### PEG-10/Lauryl Dimethicone Crosspolymer,

### PEG-12 Dimethicone Crosspolymer,

### PEG-10 Dimethicone Crosspolymer,

### PEG-10 Dimethicone/Vinyl Dimethicone Crosspolymer,

### PEG-12 Dimethicone/PPG-20 Crosspolymer,

and mixtures thereof.

They are especially sold under the KSG® names by the company Shin-Etsu:

KSG-210® INCI name: Dimethicone and Dimethicone/PEG-10/15-Crosspolymer;
KSG-310® INCI name: PEG-15/Lauryl Dimethicone Crosspolymer and Mineral oil;
KSG-320® INCI name: PEG-15/Lauryl Dimethicone Crosspolymer and Isododecane;
KSG-330® INCI name: PEG-15/Lauryl Dimethicone Crosspolymer and Triethylhexanoin;
KSG-340® INCI name: Squalane and PEG-15/Lauryl Dimethicone Crosspolymer.

They are especially sold by the company Dow Corning under the name Dow Corning 9011 Silicone Elastomer Blend®; INCI name: Cyclopentasiloxane and PEG-12 Dimethicone Crosspolymer.

Mention may also be made of the product sold under the name Dow Corning EL-7040 Hydro Elastomer Blend® by the company Dow Corning for the compound which has the INCI name: PEG-12 Dimethicone/PPG-20 Crosspolymer.

b) Polyglycerolated Silicone Elastomers

The polyglycerolated silicone elastomer is an elastomeric crosslinked organopolysiloxane that may be obtained by a crosslinking addition reaction of diorganopolysiloxane containing at least one hydrogen bonded to the silicon and of polyglycerolated compounds having ethylenically unsaturated groups, especially in the presence of a platinum catalyst.

Preferably, the elastomeric crosslinked organopolysiloxane is obtained by a crosslinking addition reaction (A) of diorganopolysiloxane containing at least two hydrogens each bonded to a silicon, and (B) of glycerolated compounds containing at least two ethylenically unsaturated groups, in particular in the presence (C) of a platinum catalyst.

In particular, the organopolysiloxane may be obtained by reaction of a dimethylvinylsiloxy-terminated polyglycerolated compound and of trimethylsiloxy-terminated methylhydropolysiloxane, in the presence of a platinum catalyst.

Compound (A) is the base reagent for the formation of elastomeric organopolysiloxane, and the crosslinking is performed by addition reaction of compound (A) with compound (B) in the presence of catalyst (C).

Compound (A) is in particular an organopolysiloxane containing at least two hydrogen atoms bonded to different silicon atoms in each molecule.

Compound (A) may have any molecular structure, especially a linear-chain or branched-chain structure or a cyclic structure.

Compound (A) may have a viscosity at 25° C. ranging from 1 to 50 000 centistokes, especially so as to be readily miscible with compound (B).

The organic groups bonded to the silicon atoms of compound (A) may be alkyl groups containing from 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group. Preferably, said organic group is chosen from methyl, phenyl and lauryl groups.

Compound (A) may thus be chosen from trimethylsiloxy-terminated methylhydropolysiloxanes, trimethylsiloxy-terminated dimethylsiloxane/methylhydrosiloxane copolymers, dimethylsiloxane/methylhydrosiloxane cyclic copolymers, and trimethylsiloxy-terminateddimethylsiloxane/methylhydrosiloxane/laurylmethylsiloxanecopolymers.

Compound (B) may be a polyglycerolated compound corresponding to the formula (B′) below:

CₘH₂ₘ₋₁—O-[Gly]ₙ-CₘH₂ₘ₋₁ (B′)

in which m is an integer ranging from 2 to 6, n is an integer ranging from 2 to 200, preferably ranging from 2 to 100, preferably ranging from 2 to 50, preferably n ranging from 2 to 20, preferably ranging from 2 to 10 and preferentially ranging from 2 to 5, and in particular n is equal to 3; Gly denotes:

—CH₂—CH(OH)—CH₂—O— or —CH₂—CH(CH₂OH)—O—

Advantageously, the sum of the number of ethylenic groups per molecule of compound (B) and of the number of hydrogen atoms bonded to silicon atoms per molecule of compound (A) is at least 4.

It is advantageous for compound (A) to be added in an amount such that the molecular ratio between the total amount of hydrogen atoms bonded to silicon atoms in compound (A) and the total amount of all the ethylenically unsaturated groups in compound (B) is within the range from 1/1 to 20/1.

Compound (C) is the catalyst for the crosslinking reaction, and is especially chloroplatinic acid, chloroplatinic acid-olefin complexes, chloroplatinic acid-alkenylsiloxane complexes, chloroplatinic acid-diketone complexes, platinum black and platinum on a support.

Catalyst (C) is preferably added in an amount of from 0.1 to 1000 parts by weight and better still from 1 to 100 parts by weight, as clean platinum metal, per 1000 parts by weight of the total amount of compounds (A) and (B).

The polyglycerolated silicone elastomer according to the invention is generally mixed with at least one hydrocarbon-based oil and/or one silicone oil to form a gel.

In these gels, the polyglycerolated elastomer is often in the form of non-spherical particles.

Such elastomers are described in particular in patent application WO 2004/024798.

Use may be made, as polyglycerolated silicone elastomers, of the following compounds having the INCI name:

Dimethicone/polyglycerin-3 crosspolymer,
Lauryl dimethicone/polyglycerin-3 crosspolymer,
and mixtures thereof.

They are especially sold by the company Shin-Etsu under the following names:

KSG-710®; INCI name: Dimethicone/Polyglycerin-3 Crosspolymer and Dimethicone;
KSG-810®; INCI name: Mineral Oil and Lauryl Dimethicone/Polyglycerin-3 Crosspolymer;
KSG-820®; INCI name: Isododecane and Lauryl Dimethicone/Polyglycerin-3 Crosspolymer;
KSG-830®; INCI name: Triethylhexanoin and Lauryl Dimethicone/Polyglycerin-3 Crosspolymer;
KSG-840®; INCI name: Squalane and Lauryl Dimethicone/Polyglycerin-3 Crosspolymer.

According to one particular form of the invention, the W/O emulsifying surfactant will be chosen from:

i) mixtures of alkyl polyglycosides and of fatty alcohol, in particular the mixture of octyldodecanol and of octyldodecyl xyloside sold under the trade name Fluidanov 20 X® from the company SEPPIC, having the INCI name Octyldodecanol (and) Octyldodecyl Xyloside;
ii) polyethylene glycol esters, such as PEG-30 dipolyhydroxystearate, such as the product sold under the name Cithrol DPHS-SO-(MV) by the company Croda;
iii) and mixtures thereof, and more particularly a mixture containing Octyldodecanol (and) Octyldecyl Xyloside and PEG-30 Dipolyhydroxystearate.

### Additives

In a known manner, the cosmetic composition can also contain adjuvants which are customary in the cosmetics field, such as hydrophilic or lipophilic gelling agents, hydrophilic or lipophilic additives, sunscreens, preservatives, antioxidants, solvents, fragrances, and also emollients, stabilizers, moisturizing agents, vitamins, desquamating agents, depigmenting agents, bactericides, polymers, a fatty phase structuring agent, in particular chosen from waxes, pasty compounds, mineral or organic lipophilic gelling agents; organic or inorganic fillers; thickeners or suspending agents.

The amounts of these various adjuvants are those conventionally used in the cosmetics field, and range, for example, from about 0.01% to 10% of the total weight of the composition. Depending on their nature, these adjuvants may be introduced into the fatty phase, into the aqueous phase and/or into lipid spherules.

Needless to say, those skilled in the art will take care to select this or these optional additional compounds such that the advantageous properties intrinsically associated with the cosmetic composition in accordance with the invention are not, or are not substantially, adversely affected by the envisaged addition(s).

### Additional Colorants

A composition according to the invention may also comprise at least one additional colorant, preferably in a proportion of at least 0.01% by weight relative to the total weight of the composition.

For obvious reasons, this amount is liable to vary significantly with regard to the intensity of the desired colour effect and of the colour intensity afforded by the colorants under consideration, and its adjustment clearly falls within the competence of those skilled in the art.

The additional colorants that are suitable for use in the invention may be water-soluble, but may also be liposoluble.

For the purposes of the invention, the term “water-soluble colorant” is intended to mean any natural or synthetic, generally organic compound, which is soluble in an aqueous phase or water-miscible solvents and which is capable of imparting colour.

As water-soluble dyes that are suitable for use in the invention, mention may be made especially of synthetic or natural water-soluble dyes, for instance FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanine (beetroot), carmine, copper chlorophylline, methylene blue, anthocyanins (enocianin, black carrot, hibiscus and elder), caramel and riboflavin.

The water-soluble dyes are, for example, beetroot juice and caramel.

For the purposes of the invention, the term “liposoluble colorant” is intended to mean any natural or synthetic, generally organic compound, which is soluble in an oily phase or in solvents that are miscible with a fatty substance, and which is capable of imparting colour.

As liposoluble dyes that are suitable for use in the invention, mention may be made especially of synthetic or natural liposoluble dyes, for instance DC Red 17, DC Red 21, DC Red 27, DC Green 6, DC Yellow 11, DC Violet 2, DC Orange 5, Sudan red, carotenes (β-carotene, lycopene), xanthophylls (capsanthin, capsorubin, lutein), palm oil, Sudan brown, quinoline yellow, annatto and curcumin.

### Moisturizing Agents

Preferably, the composition contains at least one moisturizing agent.

The moisturizing agent(s) can be present in the composition in a content ranging from 0.1% to 30% by weight, especially from 1% to 20% by weight, relative to the total weight of said composition.

Preferably, the moisturizing agent is glycerol.

### Thickeners and Suspending Agents

The thickeners may be chosen from carboxyvinyl polymers, such as Carbopols® (Carbomers) and the Pemulens® (acrylate/C10-C30 alkyl acrylate copolymer); polyacrylamides, for instance the crosslinked copolymers sold under the names Sepigel 305 (INCI name: Polyacrylamide/C13-14 Isoparaffin/Laureth 7) or Simulgel 600® (INCI name: Acrylamide/Sodium acryloyldimethyl taurate copolymer/Isohexadecane/Polysorbate 80) by SEPPIC; optionally crosslinked and/or neutralized polymers and copolymers of 2-acrylamido-2-methylpropanesulfonic acid, such as the poly(2-acrylamido-2-methylpropanesulfonic acid) sold by Hoechst under the trade name Hostacerin AMPS® (INCI name: Ammonium polyacryloyldimethyl taurate) or Simulgel 800®, sold by SEPPIC (CTFA name: Sodium polyacrylolyldimethyl taurate/Polysorbate 80/Sorbitan oleate) or Simulgel EG® sold by the company SEPPIC (INCI name: Sodium acrylate/sodium acrylolyldimethyl taurate (and) Isohexadecane (and) polysorbate 80; copolymers of 2-acrylamido-2-methylpropanesulfonic acid and of hydroxyethyl acrylate, such as Simulgel NS® and Sepinov EMT 10® sold by the company SEPPIC; cellulose-based derivatives such as hydroxyethylcellulose, cetylhydroxyethylcelIulose; polysaccharides and in particular gums such as xanthan gum, hydroxypropyl guar gums; silicas, for instance Bentone Gel MOI® sold by the company NL Industries, or Veegum Ultra® sold by the company Polyplastic.

### Cosmetic Applications

According to one embodiment, a composition of the invention can advantageously be in the form of a composition for caring for keratin materials such as the skin of the body or of the face, in particular of the face and/or of the area around the eyes, for making uniform the relief and/or colour imperfections, with a natural result, in particular for camouflaging and/or smoothing out the relief imperfections of the skin, such as pores, wrinkles and/or fine lines, and/or making uniform the complexion of face and/or neck skin, in particular correcting diffuse marks or redness of the skin and/or correcting redness of the area around the eyes, or the dark circles under the eyes.

According to one embodiment, a composition of invention can advantageously be in form of a makeup and/or care product for camouflaging and/or smoothing out the relief and/or colour imperfections of the face or of the body of the hands, for instance a foundation, and more particularly creams of the “BB Cream” or “CC Cream” type.

According to one embodiment, a composition of the invention may advantageously be in the form of a makeup and/or care product for the area around the eyes, the dark circles under the eyes, or the eyelids, such as an eyeshadow, or a concealer product.

According to one embodiment, a composition of the invention may advantageously be in the form of a lip makeup and/or care composition for camouflaging and/or smoothing out relief and colour imperfections.

Such compositions are in particular prepared according to the general knowledge of those skilled in the art.

The expression “between . . . and . . . ” should be understood as meaning limits excluded, unless otherwise specified. The expression “ranging from . . . to . . . ” should be understood as meaning limits included, unless otherwise specified.

The invention is illustrated in greater detail by the examples and figures presented below.

Unless otherwise indicated, the amounts shown are expressed as weight percentages.

### EXAMPLES

### Example 1: Corrective Day Care Cream (Oil-in-Water Emulsion)

| | | Concentration |
|---|---|---|
| Phase | INCI name | % by weight |
| A | BEESWAX | 1 |
| | PEG-30 | 1.5 |
| | DIPOLYHYDROXYSTEARATE | |
| | (CITHROL DPHS-SO-(MV) ®) | |
| | CETYL ESTERS (and) CETYL | 0.5 |
| | ESTERS (CRODAMOL MS-PA-(MH ®)) | |
| | OCTYLDODECANOL (and) | 2 |
| | OCTYLDODECYL XYLOSIDE | |
| | (FLUIDANOV 20X ®) | |
| | DICAPRYLYL ETHER | 1.5 |
| | DIMETHICONE | 1 |
| | ISOPROPYL PALMITATE | 1.5 |
| B1 | Water | qs 100 |
| | PHENOXYETHANOL | 0.5 |
| | CAPRYLYL GLYCOL | 0.3 |
| | YELLOW 6 | 0.001 |
| | RED 4 | 0.00006 |
| B2 | SODIUM ACRYLATE/SODIUM | 1.9 |
| | ACRYLOYLDIMETHYL TAURATE | |
| | COPOLYMER (and) | |
| | ISOHEXADECANE (and) | |
| | POLYSORBATE 80 | |
| | (SIMULGEL EG ®) | |
| C | GLYCEROL | 15 |
| | Water | 10 |
| D | CELLULOSE BEADS | 5 |
| | (CELLULOBEADS USF) | |
| | BORON NITRIDE | 1.5 |
| | (SOFTOUCH CCS | |
| | 102 ®) | |
| | TITANIUM DIOXIDE | 1 |
| | (and) MICA (TIMIRON | |
| | SILK RED ®) | |
| E | DIMETHICONE (and) | 0.5 |
| | DIMETHICONOL | |
| | FRAGRANCE | 0.1 |
| F | DENATURED ALCOHOL | 5 |

### Preparation Protocol

First of all, the aqueous internal phase gelled with Simulgel EG® was prepared at a temperature of 55° C. (B1+B32) with a Rayneri mixer.

The emulsion was then formed by adding the aqueous phase A heated to 80 or 85° C. Phase C was added at 1000 rpm.

The fillers and the nacres were then added to the preparation at 50° C.

At ambient temperature (25° C.), phases E and F were added at 120 rpm.

This composition 1 of the invention was compared, in terms of correction of the relief, of satin effect and of radiant effect, to a reference composition 2 on the care products market: Corrector Hydrating Cream Perfect Skin®—Sublimist—L'OREAL PARIS having as list of ingredients:

AQUA (WATER), DIMETHICONE, GLYCERIN, ISOPROPYL ISOSTEARATE, PENTAERYTHRITYL TETRAETHYLHEXANOATE, OCTYLDODECANOL, CETYL ALCOHOL, SILICA (NANO)/SILICA, BEHENYL ALCOHOL, TALC, PTFE, POLYETHYLENE, EPERUA FALCATA BARK EXTRACT, PEG-100 STEARATE, STEARIC ACID, STEARYL ALCOHOL, CARBOMER, ARACHIDYL ALCOHOL, DIMETHICONE/VINYL DIMETHICONE CROSSPOLYMER, CETEARYL ALCOHOL, CETEARYL GLUCOSIDE, SODIUM HYALURONATE, SODIUM HYDROXIDE, PALMITIC ACID, ADENOSINE, POLOXAMER 338, AMMONIUM POLYACRYLDIMETHYLTAURAMIDE/AMMONIUM POLYACRYLOYLDIMETHYL TAURATE, DISODIUM EDTA, CAPRYLOYL SALICYLIC ACID, CAPRYLYL GLYCOL, LENS ESCULENTA (LENTIL) SEED EXTRACT, DEXTRIN, PHENOXYETHANOL, CI 16035 (RED 40), CI 17200 (RED 3), LINALOOL, ALPHA-ISOMETHYL IONONE, LIMONENE, CITRAL, CITRONELLOL, FRAGRANCE, F.I.L B159063/2.

### Sensory Tests (In Vivo)

A qualitative in vivo test was carried out on a panel of 12 women, all regular users of day cream with a glowing effect; normal to dry skin, with very light to medium complexions, with slight imperfections (except redness).

The 12 women are divided up into two age groups: half are between 30 and 36 years old and the other half are between 37 and 45 years old. They are also divided up equally according to their skin type: 4 women having normal skin, 4 having mixed skin and 4 having dry skin. They exhibit:

All the women exhibit fine lines (more or less accentuated depending on age and skin nature of the women). None exhibits localized redness, in particular on the cheeks and/or the top of the forehead.

The test was carried out at home on each of the two formulations 1 and 2 for 3 days.

### Results

All of the evaluating women found that formula 1 according to the invention produced an even and uniform complexion, and a visual smoothing out effect reducing the appearance of fine lines while at the same time depositing a satiny lightness on the entire face.

All of the evaluating women found that reference formula 2 on the face care market for relief correction produced an even and uniform complexion, and a visual smoothing out effect reducing the appearance of fine lines, but, contrary to example 1 of the invention, gave a powdered matt rendering at the surface and a lack of luminosity.

### Soft-Focus Corrective Effect Tests (In Vitro)

The soft-focus corrective effect of each of Examples 1 and 2 was measured. Said soft-focus effect is characterized by haze and transparency (transmittance TH) measurements. “Haze” corresponds to the percentage of light scattered relative to the total transmittance according to standard ASTM D 1003 (Standard Test Method for Haze and Luminous Transmittance of Transparent Plastics).

25 μm films of composition were applied to 50 μm polyethylene (PE) films. The film was then measured after 1 hour of drying at ambient temperature (25° C.). Finally, the film was placed in the machine and transparency and haze measurements were carried out.

### Results

The results are summarized in the following table:

| | Transparency | |
|---|---|---|
| Composition tested | (Transmittance TH) | Haze |
| Example 1 (invention) | 92.5% | 92.13% |
| Reference Example 2 | 91.8% | 61.4% |

It results from this that the composition of Example 1 according to the invention showed good imperfection correction performance levels, an even and uniform complexion and a radiant satin effect, contrary to Reference Example 2. Furthermore, Example 1 showed a better soft-focus corrective effect, which results in a haze that is substantially higher compared with Reference Example 2.

1: A composition, comprising:
in a physiologically acceptable medium;
a) cellulose particles;
b) boron nitride particles; and
c) nacre particles having a mean size of less than 20.0 μm.

2: The composition according to claim 1, wherein the cellulose particles are in the form of spherical particles.

3: The composition according to claim 1 wherein the cellulose particles have a mean size of from 1 to 20 μm.

4: The composition according to claim 1, wherein the cellulose particles are present in a content ranging from 0.5% to 10% relative to the total weight of the composition.

5: The composition according to claim 1, wherein the boron nitride particles have a mean size of from 5 to 50 μm.

6: The composition according to claim 1, wherein the boron nitride particles have a platelet form.

7: The composition according to claim 1, wherein the boron nitride particles are present in a content ranging from 0.1% to 5% by weight relative to the total weight of the composition.

8: The composition according to claim 1, wherein the nacres are present in a content ranging from 0.1% to 3% by weight relative to the total weight of the composition.

9: The composition according to claim 1, wherein the nacre particles are selected from the group consisting of:
i) natural or synthetic micas (fluorphlogopite) covered with at least one layer of titanium dioxide (titanium mica), said titanium micas being covered with at least one material selected from the group consisting of iron oxides, bismuth oxychloride, chromium oxide, and organic dyes;
ii) natural or synthetic micas covered with at least one layer of bismuth oxychloride (bismuth oxychloride-mica), said bismuth oxychloride-micas being covered with at least one material selected from the group consisting of iron oxides, chromium oxide, chromium hydroxide, ultramarines, and organic dyes;
iii) nacres having silica or alumina as substrate, said substrate being covered with at least one layer constituted of at least one metal oxide, elected from the group consisting of titanium oxides and iron oxides; and
iv) mixtures thereof.

10: The composition according to claim 1, wherein the composition is in water-in-oil emulsion form.

11: The composition according to claim 10, wherein the composition further comprises at least one W/O emulsifying surfactant.

12: The composition according to claim 11, wherein the W/O emulsifying surfactant is selected from the group consisting of:
i) mixtures of alkyl polyglycosides and of fatty alcohol;
ii) polyethylene glycol esters;
iii) and mixtures thereof.

13: The composition according to claim 1, further comprising at least one moisturizing agent.

14: A process for cosmetic treatment of keratin materials, comprising application to the keratin materials of a composition according to claim 1.

15: The composition according to claim 11, wherein the at least one W/O emulsifying surfactant is non-ionic.

16: The composition according to claim 12 wherein the W/O emulsifying surfactant is a mixture containing Octyldodecanol (and) Octyldodecyl Xyloside and PEG-30 Dipolyhydroxystearate.
